(19) United States
(12) Patent Application Publication
(10) Pub. No.: US 2005/0148600 A1
(43) Pub. Date: Jul. 7, 2005

(54) Methods for the treatment of obesity

(75) Inventor: Jack W. Singer, Seattle, WA (US)

Correspondence Address: DONALD W. WYATT;CELL THERAPEUTICS, INC., 501 ELLIOTT AVENUE WEST, #400, SEATTLE, WA, 98119, US

(73) Assignee: Cell Therapeutics, Inc.

(21) Appl. No.: 10/925,722

(22) Filed: Aug. 25, 2004

Related U.S. Application Data
(60) Provisional application No. 60/499,907, filed on Sep. 2, 2003.

(51) Int. Cl. A61K 31/522; A61K 31/44

(52) U.S. Cl. 514/256; 514/332

(57) ABSTRACT

Methods are provided for treating or preventing obesity by inhibiting the activity of lysophosphatidic acyltransferase β (LPAAT-β).

### CROSS-REFERENCE TO RELATED APPLICATION

This application claims the benefit of U.S. Provisional Application No. 60/499,907, filed Sep. 2, 2003, which application is incorporated by reference herein in its entirety.

### BACKGROUND OF THE INVENTION

1. Field of the Invention

The present invention is generally directed toward treating or preventing obesity. This invention is more particularly related to treating or preventing obesity by inhibiting the activity of lysophosphatidic acid acyltransferase β (LPAAT-β).

2. Description of the Related Art

Lysophosphatidic acid acyltransferase (LPAAT) catalyzes the acylation of lysophosphatidic acid (LPA) to phosphatidic acid. LPA is the simplest glycerophospholipid, consisting of a glycerol molecule, a phosphate group, and a fatty acyl chain. LPAAT adds a second fatty acyl chain to LPA, producing phosphatidic acid (PA). PA is the precursor molecule for certain phosphoglycerides, such as phosphatidylinositol, and diacylglycerols, which are necessary for the production of other phosphoglycerides, such as phosphatidylcholine, and for triacylglycerols, which are essential biological fuel molecules.

In addition to being a crucial precursor molecule in biosynthetic reactions, LPA has been added to the list of intercellular lipid messenger molecules. LPA interacts with G protein-coupled receptors, coupling to various independent effector pathways including inhibition of adenylate cyclase, stimulation of phospholipase C, activation of MAP kinases, and activation of the small GTP-binding proteins Ras and Rho (Moolenaar, J. Biol. Chem. 28:1294 (1995)). The physiological effects of LPA have not been fully characterized as yet. However, one of the physiological effects that is known is that LPA promotes the growth and invasion of tumor cells (Mills and Moolenaar, Nat. Cancer Rev. 3: 582 (2003)).

Like LPA, PA is also a messenger molecule. PA is a key messenger in a common signaling pathway activated by proinflammatory mediators such as interleukin-1β, tumor necrosis factor α, platelet activating factor, and lipid A (Bursten et al., Am. J. Physiol. 262:C328 (1992); Bursten et al., J. Biol. Chem. 255:20732 (1991); Kester, J. Cell Physiol. 156:317 (1993)). PA has been implicated in mitogenesis of several cell lines (English, Cell Signal 8:341 (1996)). PA level has been found to be increased in either ras or fps transformed cell lines compared to the parental Rat2 fibroblast cell line (Martin et al., Oncogene 14:1571 (1997)). Thus, LPAAT, as an enzyme that regulates PA content in cells, may play a role in cancer, and may also mediate inflammatory responses to various proinflammatory agents.

LPAAT exists in an LPAAT-α form and an LPAAT-β form. Northern blot analysis shows that LPAAT-(X is expressed in all human tissues tested with the highest expression level found in skeletal muscle (West et al., DNA Cell Biol. 16:691 (1997)). The uniformity of LPAAT-α expression has also been found in additional tissues such as prostate, testis, ovary, small intestine, and colon (Stamps et al., Biochem. J. 326:455 (1997)) as well as in mouse tissues (Kume et al., Biochem. Biophys. Res. Commun. 237:663 (1997)). A 2 kb and a 1.3 kb forms, possibly due to alternative utilization of polyadenylation signals at the 3′-UTR, have been found in murine LPAAT-α mRNA (Kume et al., Biochem. Biophys. Res. Commun 237:663 (1997)), whereas only one major human LPAAT-α mRNA of 2 kb in size has been detected by Northern analysis (West et al., DNA Cell Biol. 16:691 (1997); Stamps et al., Biochem. J. 326:455 (1997)).

In contrast, LPAAT-β demonstrates a distinct tissue distribution of mRNA expression (West et al., DNA Cell Biol. 16:691 (1997)). LPAAT-β is most highly expressed in liver and heart tissues. LPAAT-β is also expressed at moderate levels in pancreas, lung, skeletal muscle, kidney, spleen, and bone marrow; and at low levels in thymus, brain and placenta. This differential pattern of LPAAT-β expression has been confirmed independently (Eberhardt et al., J. Biol. Chem. 272:20299 (1997)) with the only discrepancy being that high level, instead of moderate level, of LPAAT-β has been detected in pancreas, possibly due to slight lot variations in commercial RNA blots (Clontech, Palo Alto, Calif.). In addition, moderate LPAAT-β expression has been found in prostate, testis, ovary, small intestine, and colon with the small intestine containing relatively higher amounts (Eberhardt et al., J. Biol. Chem. 272:20299 (1997)). Within various brain sections, high expression has been found in the subthalamic nucleus and spinal cord; and least in the cerebellum, caudate nucleus, corpus callosum, and hippocampus. LPAAT-β can also be detected in myeloid cell lines THP-1, BL-60, and U937 with the mRNA levels remaining the same with or without phorbal-ester treatment. The size difference between human LPAAT-α and LPAAT-β mRNA is consistent with the sequence data, in which LPAAT-α has a longer 3′-UTR. The differential tissue expression pattern of LPAAT-α and LPAAT-β mRNA would suggest these two genes are regulated differently and are likely to have independent functions. Therefore, a desirable feature in compounds that inhibit LPAAT activity is that they are specific in inhibiting one isoform of the enzyme over the other (i.e., LPAAT-β over LPAAT-α).

Obesity is a problem in a number of countries throughout the world, and in the United States in particular. In the United States, it appears that obesity is on the rise, especially for children. Treatments for obesity are desired.

Thus, there is a need in the art for improved compositions and methods related to treating obesity. The present invention fills this need, and further provides other related advantages.

### BRIEF SUMMARY OF THE INVENTION

Briefly stated, the present invention provides a variety of methods to treat or prevent obesity in warm-blooded animals, such as humans. More specifically, a compound that inhibits (i.e., decreases) the activity of LPAAT-β is administered to treat or prevent obesity. The compound may be a variety of forms (e.g., salts thereof), and may be combined with a pharmaceutical carrier or diluent to form a pharmaceutical composition for use in the methods of the present invention.

In one embodiment, the present invention provides a method of treating obesity comprising administering to a patient in need thereof in an amount effective to treat obesity a compound that inhibits lysophosphatidic acid acyltransferase β (LPAAT-β). The step of administering may be repeated one or more times. In a preferred embodiment, the step of administering is repeated one or more times.

In another embodiment, the present invention provides a method of preventing obesity comprising administering to an individual with increased risk of obesity in an amount effective to prevent obesity a compound that inhibits lysophosphatidic acid acyltransferase β (LPAAT-β). The step of administering may be repeated one or more times. In a preferred embodiment, the step of administering is repeated one or more times.

These and other aspects of the present invention will bercome evident upon reference to the following detailed description.

### DETAILED DESCRIPTION OF THE INVENTION

As noted above, the present invention is directed toward methods to treat or prevent obesity in warm-blooded animals, such as humans. In particular, the invention relates to the treatment or prevention of obesity by inhibiting the activity of lysophosphatidic acid acyltransferase β (LPAAT-β). As used herein, the phrase “inhibiting the activity of LPAAT-β” refers to decreasing the enzymatic activity of LPAAT-β from its level prior to administration of an LPAAT-β inhibitor. The decrease in activity may be partial or complete.

The activity of LPAAT-β is inhibited using an LPAAT-β inhibitor compound. A variety of molecules are LPAAT-β inhibitor compounds, such as antibodies to LPAAT-β, compounds that interfere with the production (e.g., expression) of LPAAT-β, and small organic compounds that interfere with the enzymatic function of LPAAT-β. Examples of such small organic compounds include aryl triazines (U.S. Patent Application Publication No. US 2003-0153570), pyrimidines (e.g., U.S. Patent Application Ser. No. 60/460,776), and pyridines (e.g., U.S. Patent Application Ser. No. 60/460,782). These examples of LPAAT-β inhibitors are provided to exemplify, and not to limit, the present invention.

Compounds useful in the present invention may be prepared and used in a salt form, i.e., as a physiologically acceptable salt. The phrase “physiologically acceptable salt” refers to those salts that retain the biological effectiveness and properties of the particular compound. Physiologically acceptable salts are often useful because they may have improved stability and/or solubility in pharmaceutical compositions over the free base form or free acid form of the compound. A physiologically acceptable salt may be obtained by reaction of a free base with an inorganic acid such as hydrochloric acid, hydrobromic acid, nitric acid, phosphoric acid, sulfuric acid, and perchloric acid and the like, or with an organic acid such as acetic acid, oxalic acid, malic acid, maleic acid, methanesulfonic acid, ethanesulfonic acid, p-toluenesulfonic acid, salicylic acid, tartaric acid, citric acid, succinic acid or malonic acid, and the like. A physiologically acceptable salt may also be obtained by reaction of a free acid with a base such as sodium, potassium or lithium hydroxide, bicarbonate or carbonate, and the like.

It may be advantageous for certain uses to enhance the solubility and/or bioavailability of one or more of the compounds useful in the present invention. This may be accomplished, for example, by the addition of one or more substituents to the compound. For example, the addition of hydrophilic groups, such as hydroxyl groups, may be advantageous. Other substituents for enhancing solubility and/or bioavailability include amino acids (e.g., polyglutamate or polylysine), di-peptides, polymers (e.g., PEG or POG), monocarboxylic acids (e.g., hemi-succinate), and esters. Any group that enhances solubility and/or bioavailability of a compound useful in the present invention may be used, provided that the group does not significantly impair the relevant biological property of the compound, i.e., as an inhibitor of LPAAT-β activity.

It may be advantageous for certain uses to prepare a compound (or physiologically acceptable salt thereof) as a “prodrug.” As used herein, the term “compound” encompasses a prodrug form of the parent compound. “Prodrug” herein refers to a chemical substance that is converted into the parent compound in vivo. Prodrugs often are useful because, in some situations, they may be easier to administer than the parent compound. They may, for instance, be bioavailable by oral administration whereas the parent compound is not. The prodrug may also have improved solubility in pharmaceutical compositions over the parent compound. An example of a prodrug would be a parent compound useful in the present invention which is administered as an ester (the “prodrug”) to facilitate transmittal across a cell membrane where water solubility is detrimental to mobility. The ester is then metabolically hydrolyzed to the carboxylic acid, the active entity, once inside the cell where water solubility is beneficial. Such a prodrug is generally inactive (or less active) until converted to the active form.

Pharmaceutical compositions of the compounds and the physiologically acceptable salts thereof are preferred embodiments in the methods of this invention. Pharmaceutical compositions of the compounds useful in the present invention (i.e., compounds and salts thereof as described above) may be manufactured by processes well known in the art; e.g., by means of conventional mixing, dissolving, granulating, dragee-making, levigating, emulsifying, encapsulating, entrapping or lyophilizing processes.

Pharmaceutical compositions may be formulated in a conventional manner using one or more physiologically acceptable carriers or diluents. Proper formulation is generally dependent upon the route of administration chosen. The compounds useful in the present invention may be formulated such that the formulation comprises a single compound or a mixture of two or more compounds. Alternatively, one or more compounds may be formulated with one or more other agents which are relevant to a symptom or cause of obesity.

For injection, the compounds useful in the invention may be formulated as sterile aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with physiologically acceptable carriers well known in the art. Such carriers enable the compounds useful in the invention to be formulated as tablets, pills, dragees, capsules, liquids, gels, syrups, slurries, suspensions and the like, for oral ingestion by a patient to be treated. Pharmaceutical preparations for oral use can be made with the use of a solid carrier or diluent, optionally grinding the resulting mixture, and processing the mixture of granules, after adding suitable auxiliaries, if desired, to obtain tablets or dragee cores. Suitable carriers or diluents are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the embodiments of the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoro-ethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions for parenteral administration include sterile aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation (see, for example, U.S. Pat. No. 5,702,717 for a biodegradable depot for the delivery of a drug). Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. The pharmaceutical compositions useful herein also may comprise suitable solid or gel phase carriers or diluents. Examples of such carriers or diluents include but are not limited to calcium carbonate, calcium phosphate, various sugars, starches, cellulose derivatives, gelatin, and polymers such as polyethylene glycols.

The compounds useful in the invention may be provided as physiologically acceptable salts wherein the claimed compound may form the negatively or the positively charged species. Examples of salts in which the compound forms the positively charged moiety include quaternary ammonium salts such as the hydrochloride, sulfate, carbonate, lactate, tartarate, maleate, succinate, etc. formed by the reaction of an amino group with the appropriate acid.

In the context of the present invention, the term “animal” refers to any animal, including humans and other primates, rodents (e.g., mice, rats, and guinea pigs), lagamorphs (e.g., rabbits), bovines (e.g., cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc), as well as feral or wild animals, including such animals as ungulates (e.g., deer), bear, fish, lagamorphs, rodents, birds, etc. It is not intended that the term be limited to a particular age or sex. Thus, adult and newborn subjects, as well as fetuses, whether male or female, are encompassed by the term. A preferred animal within the present invention is a mammal, with humans particularly preferred.

As disclosed herein, the present invention provides that an LPAAT-β inhibitor is administered to an animal to treat or prevent obesity. Treatment of obesity may be evaluated in a variety of ways, including one or more of weight reduction or arresting weight gain or altered body composition. Prevention of obesity if typically relevant for individuals at increased risk of obesity and complication thereof. Increased risk of obesity may be related, for example, to familial or environmental factors. Prevention of obesity may be effected for a period in an individual's maturation, or a shorter or longer time interval.

Suitable routes of administration may include oral, rectal, transmucosal or intestinal administration or intramuscular, subcutaneous, intramedullary, intrathecal, direct intraventricular, intravenous, intraperitoneal or intranasal injections.

Alternately, one may administer a compound or composition in a local rather than systemic manner, for example, via injection of the compound or composition directly into a solid tumor, often in a depot or sustained release formulation.

Furthermore, one may administer a compound or composition in a targeted drug delivery system, for example, in a liposome or conjugated to a polymer.

Compounds and compositions suitable for use in the methods of the present invention are compounds and compositions wherein the active ingredients are contained in an amount effective to achieve its intended purpose. Determination of an effective amount is well within the capability of one of ordinary skill in the art, especially in light of the disclosure provided herein.

For any compound or composition used in the methods of the invention, the effective amount or dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of LPAAT-β activity). Such information can be used to more accurately determine useful doses in humans.

Toxicity and therapeutic efficacy of the compounds described herein can be determined by standard pharmaceutical procedures in cell cultures or experimental animals for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (see, e.g., Fingl, et al., in “The Pharmacological Basis of Therapeutics,” (1975), Chapter 1, pp. 1).

Dosage amount and interval may be adjusted individually to provide plasma levels of the active moiety which are sufficient to maintain LPAAT-β inhibitory effects, or minimal effective concentration (MEC). The MEC will vary for each compound but can be estimated from in vitro data; e.g., the concentration necessary to achieve 50-90% inhibition of LPAAT-β using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

The amount of compound or composition administered will, of course, be dependent on the subject being treated, on the subject's weight, the severity of the affliction, the manner of administration and the judgment of the prescribing physician. An exemplary systemic daily dosage is about 5 to about 200 mg/kg of body weight. Normally, from about 10 to about 100 mg/kg of body weight of the compounds of the present invention, in one or more dosages per day, is effective to obtain the desired results. One of ordinary skill in the art can determine the optimal dosages and concentrations of the compounds of the preferred embodiments of the present invention with only routine experimentation.

The compounds when used in the present invention are substantially pure and preferably sterile. The phrase “substantially pure” encompasses compounds created by chemical synthesis or compounds substantially free of chemicals which may accompany the compounds in the natural state, as evidenced by thin layer chromatography (TLC) or high performance liquid chromatography (HPLC) or mass spectrometry.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating obesity comprising administering to a patient in need thereof in an amount effective to treat obesity a compound that inhibits lysophosphatidic acid acyltransferase β (LPAAT-β).

2. The method of claim 1 wherein the step of administering is repeated.

3. A method of preventing obesity comprising administering to an individual with increased risk of obesity in an amount effective to prevent obesity a compound that inhibits lysophosphatidic acid acyltransferase β (LPAAT-β).

4. The method of claim 3 wherein the step of administering is repeated.
